(19) United States
(12) Patent Application Publication
(10) Pub. No.: US 2024/0081153 A1
(43) Pub. Date: Mar. 7, 2024

(54) MODULE AND WEARABLE DEVICE

(71) Applicant: CASIO COMPUTER CO., LTD., Tokyo (JP)

(72) Inventor: Masashi SAWAI, Tokyo (JP)

(73) Assignee: CASIO COMPUTER CO., LTD., Tokyo (JP)

(21) Appl. No.: 18/460,890

(22) Filed: Sep. 5, 2023

(30) Foreign Application Priority Data
Sep. 5, 2022 (JP) 2022-140460

(51) Int. Cl. H10N 30/88; A61B 5/00; A61B 5/021; A61B 5/024; A61B 5/145; H10N 30/20

(52) U.S. Cl. CPC H10N 30/883; A61B 5/021; A61B 5/024; A61B 5/14542; A61B 5/681; A61B 5/7405; H10N 30/2047

(57) ABSTRACT

A module includes a circuit board that is loaded with a biological information obtainer, a piezoelectric sounder that overlaps the circuit board and includes a piezoelectric body; and a cushion that is provided between the circuit board and the piezoelectric sounder. The piezoelectric sounder is arranged such that a center position of a surface of the piezoelectric body is offset from a center position of a surface of the circuit board.

### CROSS-REFERENCE TO RELATED APPLICATIONS

This application is based upon and claims the benefit of priority from the prior Japanese Patent Application No. 2022-140460, filed on Sep. 5, 2022, the entire contents of which are incorporated herein by reference.

### TECHNICAL FIELD

This disclosure relates to a module and a wearable device.

### DESCRIPTION OF RELATED ART

JP2021-013404A discloses a device that is loaded with a biological information obtainer (various biological sensors, etc.) to obtain various types of information about a living body non-invasively.

### SUMMARY OF THE INVENTION

According to an aspect of the present invention, there is provided a module including:
a circuit board that is loaded with a biological information obtainer;
a piezoelectric sounder that overlaps the circuit board and includes a piezoelectric body; and
a cushion that is provided between the circuit board and the piezoelectric sounder,
wherein a center position of a surface of the piezoelectric body is offset from a center position of a surface of the circuit board.

### BRIEF DESCRIPTION OF DRAWINGS

The accompanying drawings are not intended as a definition of the limits of the invention but illustrate embodiments of the invention, and together with the general description given above and the detailed description of the embodiments given below, serve to explain the principles of the invention, wherein:

FIG. 1 is a cross-sectional diagram of main parts of a watch in an embodiment;

FIG. 2 is an exploded perspective view of main parts arranged on a side of a case back illustrated in FIG. 1;

FIG. 3A is a plan view of a side where a sensor is loaded of a circuit board of the embodiment;

FIG. 3B is a plan view of a back side of the circuit board illustrated in FIG. 3A;

FIG. 4A is a plan view of a piezoelectric sounder viewed from a visible side of the embodiment;

FIG. 4B is a plan view of the piezoelectric sounder illustrated in FIG. 4A viewed from a non-visible side; and

FIG. 5 is a cross-sectional view of main parts of a case back and parts arranged on the case back of the embodiment.

### DETAILED DESCRIPTION

With reference to the drawings, an embodiment of a module (circuit board fixing structure) and a wearable device having the module will be described, including how the module is assembled. In the example described in the present embodiment, the wearable device is an electronic watch to be worn on a user's arm.

The embodiment described below includes various technically preferred limitations for carrying out the present invention, but the scope of the present invention is not limited to the following embodiment and illustrated examples.

### Configuration

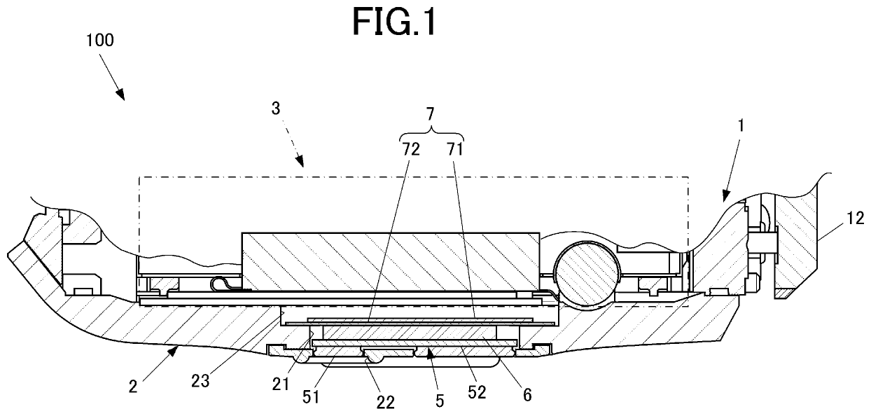

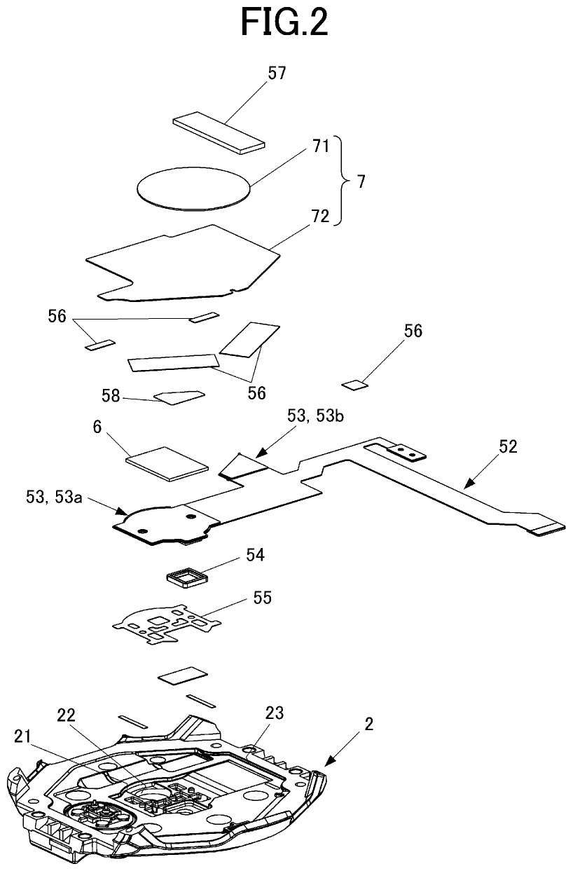

FIG. 1 is a cross-sectional diagram of main parts of a wearable device (hereinafter simply “watch”) in the present embodiment. FIG. 2 is an exploded perspective view of main parts on the side of the case back of the watch illustrated in FIG. 1.

As shown in FIG. 1, the watch 100, which is a wearable device in the present embodiment, has a device case 1.

The device case 1 in the present embodiment is formed in the shape of a short tube that is open at the top and the bottom (a front side and a back side of the watch 100). The hollow space inside the device case 1 constitutes a space to hold various parts.

The device case 1 is formed of a relatively hard synthetic resin, such as an engineering plastic or a super engineering plastic. The material for forming the device case 1 is not limited to that illustrated here. The device case 1 may be made of a metallic material, such as SUS, for example.

Conventionally, there is a device having a biological information obtainer in which the part sensing biological information (such as a biological sensor) is configured to be arranged in close proximity to the living body.

Also, there is a wearable device having a sound generator (piezoelectric sound generating unit) that generates a beep sound (buzzer sound) to inform the user of various information. In such a sound generator (piezoelectric sounder), for example, a piezoelectric body (piezoelectric element) is periodically deformed in response to application of a varying voltage, and a beep sound can be generated by the vibration of the diaphragm to which the piezoelectric body is attached.

A wearable device loaded with a biological information obtainer (various biological sensors, etc.) needs to generate a beep sound at a suitable sound pressure level that is easily audible from the viewpoint of usability and the like.

A module and a wearable device that allows the piezoelectric sounder in the device to generate beeps or the like at a suitable sound pressure level without any reduction in the sound pressure level according to such needs are described below, including how the module is assembled.

The device case 1 has various operation buttons 12 (pushbuttons, crowns, and the like) for various input operations by the user on the outside of the device case 1, as shown in FIG. 2.

Although not shown in the drawings, the device case 1 has a pair of band attachment portions where a band (not shown) can be attached at the 12 o'clock and 6 o'clock positions of the analog watch on the outer surface.

The opening on the front side of the device case 1 (a visible side of the watch) is closed by a windshield member, which is not shown in the drawings. The windshield member is a transparent member (cover member) formed of a glass material or a transparent resin material, for example. In addition, the device case 1 may have an exterior member such as a bezel (not shown in the drawings) surrounding the opening on the front side of the device case 1 (the visible side of the watch).

Although detailed illustrations and explanations are omitted, the device case 1 holds a controller, an operating part, and a battery of the watch 100, which is the wearable device.

The watch 100 having a digital display system includes a display and the like which includes a liquid crystal display panel and is arranged on the front side of the watch (the visible side, upper side in FIG. 1), as the operating part. The watch 100 having an analog display system includes hands, a gear train (a wheel mechanism) that rotates the hands, and a driving unit (a motor or the like) as the operating part instead of the above configurations. The watch 100 may be a hybrid type with both digital and analog systems, which has parts for both digital and analog configurations as the operating part.

The controller is mounted on a circuit board and the like (not shown in the drawings) and controls the display operation and the like by the operating part. The operating part and the controller are collectively referred to as a movement 3 illustrated in FIG. 1.

The opening on the back side of the device case 1 (a non-visible side of the watch) is closed by a case back 2. The case back 2 may be formed integrally with the device case 1.

The case back 2 is preferably attached to the device case 1 with a waterproof ring or the like (not shown in the drawings) between them. The case back 2 attached to the device case 1 with the waterproof ring or the like between them can close the opening on the back side of the device case 1 (the non-visible side of the watch) while ensuring waterproofness (airtightness) inside the device case 1.

As shown in FIGS. 1 and 2, a recess 21 is formed approximately in the middle of the surface of the case back 2.

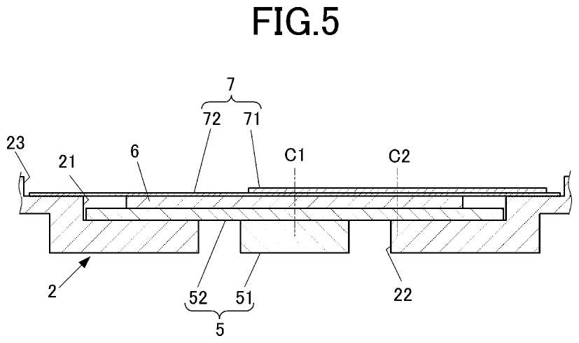

In the recess 21 is arranged a biological sensor unit 5 (see FIG. 5). The biological sensor unit 5 includes a biological sensor 51 as a biological information obtainer and a circuit board 52 loaded with the biological sensor 51.

In the recess 21 of the case back 2 is a window 22 which the biological sensor 51 aligns with when the biological sensor unit 5 is assembled.

The biological sensor 51 is a sensor that can obtain biological information of a living body, for example, a heart rate sensor, a blood pressure sensor, a blood oxygenation sensor, and the like. The biological sensor 51 is not limited to those shown in the examples here, as long as it obtains biological information. The biological sensor 51 may obtain multiple types of biological information.

The biological sensor 51 has, for example, a light-emitting part including a light emitting diode (LED) and a light-receiving part including a photodiode (PD) (both not shown).

For example, when the heart rate is obtained as the biological information, the light-emitting part of the biological sensor 51 emits light to the user's arm or the like, which is a living body. The light-emitting part emits, for example, green light that is reflected by relatively shallow portions of the skin, but is not limited thereto. The light-emitting part may be able to emit multiple types of light depending on the type of biological information to be obtained and its application.

The light-receiving part receives the reflected light that has been emitted by the light-emitting part and reflected by the arm. The light-receiving part then outputs a detection signal as detection information indicating the amount of received reflected light.

An arithmetic unit (not shown in the drawings) loaded onto the circuit board 52 or the like processes the detection information and calculates the user's heart rate and the like.

FIGS. 3A and 3B are plan views of the circuit board 52 of the biological sensor unit 5 in the present embodiment. FIG. 3A is a diagram of the circuit board 52 viewed from the back side (the non-visible side of the watch or the lower side in FIGS. 1 and 2) when the biological sensor unit 5 is assembled to the case back 2. FIG. 3A is a diagram of the circuit board 52 viewed from the front side (the visible side of the watch or the upper side in FIGS. 1 and 2) when the biological sensor unit 5 is assembled to the case back 2.

The circuit board 52 in the present embodiment is a flexible printed circuit board (hereinafter referred to as “FPC”) that can be bent and deformed. The double-dotted lines in FIGS. 3A and 3B indicate mountain folds when the biological sensor unit 5 is assembled into the case back 2. The dotted lines in FIGS. 3A and 3B indicate valley folds when the biological sensor unit 5 is assembled into the case back 2.

At one end of the circuit board 52 is provided a main circuit board body 53a loaded with the biological sensor 51. Also, a sub circuit board body 53b loaded with other circuit elements is connected to the main circuit board body 53a via the FPC.

The other end of the circuit board 52 is connected to a circuit board (main circuit board) in the movement 3, which is not shown in the drawings.

On the main circuit board body 53a is arranged a cushion 61 having a thickness equal to or greater than that of the biological sensor 51 (see FIG. 3A). The cushion 61 prevents light to and from the biological sensor 51 from leaking out.

Furthermore, a cushion 6 is attached to the surface of the main circuit board body 53a opposite to the surface where the biological sensor 51 is loaded (see FIG. 3B).

The cushion 61 and the cushion 6 are formed of a resin or the like having excellent shock absorption properties. An example of the suitably used material for the cushion 6 is a high-performance urethane foam such as Microcell polymer sheet (for example, “PORON” (registered trademark) manufactured by Rogers Inoac Corporation). The materials used to form the cushion 61 and the cushion 6 are not limited to this. The materials used to form the cushion 61 and the cushion 6 may be different from each other.

The main circuit board body 53a is arranged in the recess 21 of the case back 2 with an insulating sheet 55 on the side where the biological sensor 51 is loaded. The insulating sheet 55 is sandwiched and fixed between the main circuit board body 53a and the case back 2, for example.

On the upper side (i.e., the side opposite to the case back 2, the visible side of the watch) of the biological sensor unit 5 is arranged a piezoelectric sounder 7, with the cushion 6 between the biological sensor unit 5 and the piezoelectric sounder 7. The piezoelectric sounder 7 generates a beep sound (buzzer) to provide various notifications to the user. As shown in FIG. 2, a cushion 57 for shock absorption is arranged on the upper side (visible side) of the piezoelectric sounder 7 to protect the piezoelectric sounder 7 from shocks. The cushion 57 is preferably arranged at the outer periphery of the piezoelectric sounder 7 so as not to interfere with the vibration of the piezoelectric sounder 7 (described below).

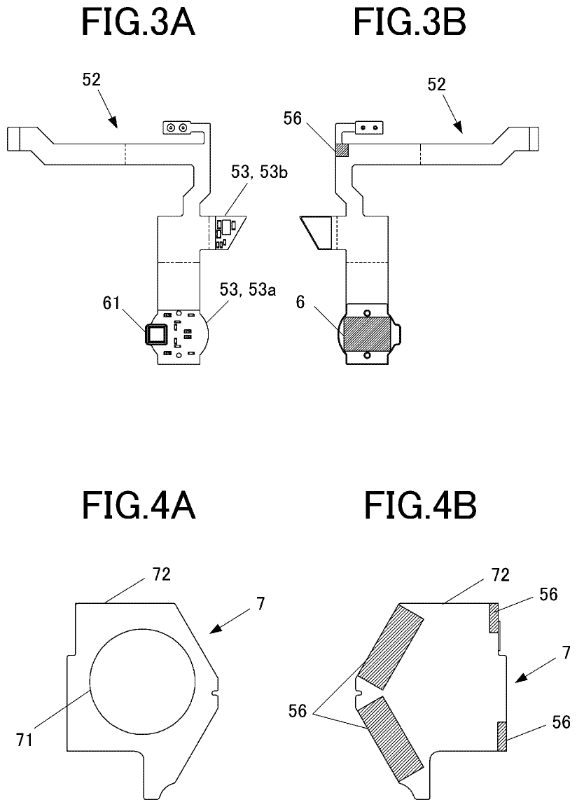

FIG. 4A is a plan view of the piezoelectric sounder 7 viewed from the visible side (the upper side in FIGS. 1 and 2). FIG. 4B is a plan view of the piezoelectric sounder 7 viewed from the non-visible side (the lower side in FIGS. 1 and 2).

When the piezoelectric sounder 7 is arranged above the biological sensor unit 5 with the cushion 6 between them, the surface of the piezoelectric sounder 7 illustrated in FIG. 4B faces the cushion 6 (see FIG. 2).

As illustrated in FIGS. 4A and 4B, the piezoelectric sounder 7 of the present embodiment has a unimorph structure in which a piezoelectric body 71 is bonded to a metal plate 72. The piezoelectric body 71 is, for example, a piezoelectric ceramic (a piezoelectric porcelain plate) on which an electrode (not shown in the drawings) is formed. The metal plate 72 is a sheet metal such as SUS, for example. The metal plate 72 is bonded to the piezoelectric body 71 to form the piezoelectric sounder 7. The metal plate 72 is not limited to SUS, but may be brass, nickel, or the like.

When voltage is applied to the piezoelectric sounder 7, the piezoelectric body 71 (piezoelectric ceramic, piezoelectric porcelain plate) is expanded, while the metal plate 72 bonded to the piezoelectric body 71 is neither expanded nor contracted. This causes the entire piezoelectric sounder 7 to bend when voltage is applied to the piezoelectric sounder 7. When the voltage applied to the piezoelectric sounder 7 is reversed, the entire piezoelectric sounder 7 bends in the opposite direction. Therefore, when a signal is input such that the direction of the voltage changes alternately, the entire piezoelectric sounder 7 changes the direction of bending alternately. In response to such signal input, the piezoelectric sounder 7 vibrates, generates sound waves, and outputs a beep sound.

Pieces of a double-sided tape 56 are affixed to the outer edge of the non-visible surface of the piezoelectric sounder 7, as illustrated in FIG. 4B. The case back 2 has a step 23 on the inner surface (the upper surface in FIG. 2) that forms a recess that fits the piezoelectric sounder 7. The piezoelectric sounder 7 is arranged within the step 23 of the case back 2. The piezoelectric sounder 7 and the case back 2 are affixed together with the double-sided tape 56.

FIG. 5 is a cross-sectional view schematically illustrating the positional relationship of the biological sensor unit 5 and the piezoelectric sounder 7 arranged inside the case back 2.

As illustrated in FIG. 5, when the biological sensor unit 5 is arranged inside the case back 2 such that the biological sensor 51 is exposed to the outside through the window 22 (directly or indirectly through glass, etc.), the circuit board 52 of the biological sensor unit 5 is arranged almost along the bottom surface of the recess 21, and the piezoelectric sounder 7 is arranged above the biological sensor unit 5 with the cushion 6 between them.

As a result, the circuit board 52 is fixed such that the biological sensor 51, which is the biological information obtainer, can face and approach an external living body (for example, an arm of a user). In other words, the piezoelectric sounder 7 functions as a fixing member that fixes the biological sensor unit 5 including the circuit board 52 to a predetermined position.

### Actions

The assembly of the watch 100 in the present embodiment includes holding of the movement 3 in the device case 1, closing of the opening on the visible side with the windshield member, and closing of the opening on the non-visible side with the case back 2.

The biological sensor unit 5 is arranged inside of the case back 2 (the side facing the inside of the device case 1). Specifically, the circuit board 52 is arranged in the recess 21 such that the biological sensor 51 is exposed through the window 22 in the case back 2. Then, the piezoelectric sounder 7 is arranged above the biological sensor unit 5 with the cushion 6 between them, and fixed to the inner surface of the case back 2 (inside the step 23 of the case back 2), etc., with the double-sided tape 56.

In the present embodiment, when the piezoelectric sounder 7 is arranged inside the step 23 of the case back 2 and overlaps the circuit board 52 (the biological sensor unit 5 including the circuit board 52), a center position C2 of a surface of the piezoelectric body 71 of the piezoelectric sounder 7 is arranged so as to be offset from a center position C1 of a surface of the circuit board 52 in a plan view (as viewed from the direction normal to the surface of the piezoelectric body 71) as illustrated in FIG. 5. That is, the center position C2 of is different from the center position C1.

The piezoelectric sounder 7 having a unimorph structure is most likely to vibrate at and near the center position C2 of the piezoelectric 71, and the vibration affects the sound pressure level of the generated sound.

Therefore, when the center C1 position of the circuit board 52 (the biological sensor unit 5 including the circuit board 52) is offset from the center position C2 of the piezoelectric body 71, the piezoelectric sounder 7, which also serves as a fixing member that fixes the circuit board 52 (the biological sensor unit 5 including the circuit board 52) at a predetermined position, can vibrate sufficiently. As a result, the piezoelectric sounder 7 can generate sound with a suitable sound pressure level with minimal reduction.

More specifically, the following action occurs.

The metal plate 72 of the piezoelectric sounder 7 is bonded and fixed to the case back 2 with the double-sided tape (not shown in FIG. 5). At least a part of the piezoelectric body 71 is arranged so as to overlap a fixing surface where the metal plate 72 to which the piezoelectric body 71 is bonded is fixed to the case back 2 in a plan view. In the present embodiment, this piezoelectric sounder 7 fixes the circuit board 52 of the biological sensor unit 5 with the cushion 6 between them. When the center position C2 of the circular piezoelectric body 71 in the piezoelectric sounder 7 is offset from the center position C1 of the circuit board 52 of the biological sensor unit 5 in a plan view, the piezoelectric sounder 7 can generate sound with a suitable sound pressure level with minimal reduction.

This is because the circuit board 52 of the biological sensor unit 5 easily bends at its center position C1 that is separated from the portion where the case back 2 and the metal plate 72 of the piezoelectric sounder 7 are fixed together with the double-sided tape in a plan view. Also, the center of the cushion 6 is almost the same as the center position C1 of the circuit board 52 of the biological sensor unit 5. Therefore, the cushion 6 bends most easily at its center, and bends upward. The piezoelectric body 71 of the piezoelectric sounder 7 oscillates up and down to generate sound. The upward bending of the cushion 6 reduces the downward bending of the piezoelectric body 71 of the piezoelectric sounder 7, thereby reducing the amplitude of the piezoelectric sounder 7. As a result, the sound pressure level of the sound emitted by the piezoelectric sounder 7 is reduced. Therefore, when the center C1 of the circuit board 52 (the biological sensor unit 5 including the circuit board 52) is offset from the center position C2 of the piezoelectric body 71, the difficulty in vibrating the piezoelectric 71 can be alleviated.

### Effects

As described above, the module of the present embodiment includes the circuit board 52, the piezoelectric sounder 7, and the cushion 6. The circuit board 52 is loaded with the biological sensor 51, which is the biological information obtainer. The piezoelectric sounder 7 overlaps the circuit board 52 and includes the piezoelectric body 71. The cushion 6 is provided between the circuit board 52 and the piezoelectric sounder 7. The piezoelectric sounder 7 is arranged such that the center position C2 of the surface of the piezoelectric body 71 is offset from the center position C1 of the surface of the circuit board 52.

The piezoelectric sounder 7 thereby fixes the biological sensor unit 5 in an appropriate position inside the case back 2 (i.e., such that the biological sensor 51 is exposed through the window 22, and the circuit board 52 is arranged in the recess 21). In this case, the center position C2 of the piezoelectric body 71, which vibrates most easily and most affects the sound pressure level of the generated sound in the piezoelectric sounder 7, is arranged so as to be offset from the center position C1 of the circuit board 52. Therefore, a sufficient sound pressure level can be secured and a beep sound with the suitable sound pressure level can be output.

In the present embodiment, the piezoelectric sounder 7 also functions as a fixing member that fixes the circuit board 52 (the biological sensor unit 5 including the circuit board 52) such that the biological sensor 51 faces and approaches an external living body.

Thus, in the present embodiment, the circuit board 52 (the biological sensor unit 5 including the circuit board 52) is fixed by the piezoelectric sounder 7 that the electronic device (the wearable device) such as the watch 100 is originally provided with. Therefore, there is no need to additionally include a part for fixing the circuit board 52. The reduction in the number of parts contributes to downsizing and weight reduction of the entire device (the watch 100) in which the module is incorporated.

The piezoelectric sounder 7 in the present embodiment has a unimorph structure in which the piezoelectric body 71 is bonded to a metal plate 72.

Such a piezoelectric sounder 7 of a relatively simple configuration realizes downsizing and weight reduction of the entire device (the watch 100) in which the module is incorporated.

In the present embodiment, the circuit board 52 (the biological sensor unit 5 including the circuit board 52) and the piezoelectric sounder 7 are arranged so as not to be in direct contact with each other.

Therefore, the biological sensor unit 5 does not interfere with the sound generation by the piezoelectric sounder 7. Thus, a sufficient sound pressure level can be secured and a beep sound with a suitable sound pressure level can be output.

In the present embodiment, the biological sensor 51, which is the biological information obtainer, includes at least one of a heart rate sensor, a blood pressure sensor, and a blood oxygenation sensor.

Therefore, the user can easily obtain biological information such as the heart rate by simply wearing the wearable device loaded with the module of the present embodiment on the arm or other portion of the body.

When the watch 100, the wearable device of the present embodiment, is loaded with the above structure of fixing the circuit board 52 (the biological sensor unit 5 including the circuit board 52), the biological sensor unit 5 can be efficiently fixed with a small number of parts. Therefore, even the device (the watch 100) that can obtain biological information such as the heart rate and the blood pressure can be made smaller and lighter.

In this case, as shown in FIG. 5, the center position C2 of the piezoelectric body 71, which vibrates most easily and most affects the sound pressure level of the generated sound in the piezoelectric sounder 7, is arranged so as to be offset from the center position C1 of the circuit board 52. Therefore, a sufficient sound pressure level can be secured and a beep sound with the suitable sound pressure level can be output.

In the present embodiment, the case back 2 is provided on the side of the device case 1 facing a living body (such as the user's arm) which is a target from which the biological information is obtained. The circuit board 52 (the biological sensor unit 5 including the circuit board 52) is arranged and secured at a predetermined position on the case back 2.

When the position of the biological sensor 51 of the biological sensor unit 5 is displaced, it is usually difficult to perform accurate sensing. However, in the present embodiment, the biological sensor 51 can be fixed in a position suitable for sensing, which enables accurate biological information to be obtained.

Although the embodiment of the present invention has been described above, it goes without saying that the present invention is not limited to such embodiment and can be changed in various ways as long as it does not depart from the gist of the present invention.

For example, in the embodiment described above, the wearable device is the watch 100, and the module (the structure that fixes the circuit board 52 of the biological sensor unit 5) is loaded onto the watch 100. However, the device loaded with the module (fixing structure of the circuit board 52) of the above embodiment can be applied is not limited to the watch 100.

The module of the above embodiment can be loaded onto a wide range of electronic devices such as various types of smartwatches and sports watches, wearable devices that obtain biological information on heartbeat, blood flow, etc. in addition to time.

Although some embodiments of the present invention have been described and illustrated in detail, the disclosed embodiments are made for purposes of not limitation but illustration and example only. The scope of the present invention should be interpreted by terms of the appended claims.

1. A module comprising:
a circuit board that is loaded with a biological information obtainer;
a piezoelectric sounder that overlaps the circuit board and includes a piezoelectric body; and
a cushion that is provided between the circuit board and the piezoelectric sounder,
wherein the piezoelectric sounder is arranged such that a center position of a surface of the piezoelectric body is offset from a center position of a surface of the circuit board.

2. The module according to claim 1,
wherein the piezoelectric sounder fixes the circuit board such that the biological information obtainer faces and approaches an external living body.

3. The module according to claim 1,
wherein the piezoelectric sounder has a unimorph structure in which the piezoelectric body is bonded to a metal plate.

4. The module according to claim 2,
wherein the piezoelectric sounder has a unimorph structure in which the piezoelectric body is bonded to a metal plate.

5. The module according to claim 1,
wherein the circuit board and the piezoelectric sounder are not in direct contact with each other.

6. The module according to claim 2,
wherein the circuit board and the piezoelectric sounder are not in direct contact with each other.

7. The module according to claim 3,
wherein the circuit board and the piezoelectric sounder are not in direct contact with each other.

8. The module according to claim 4,
wherein the circuit board and the piezoelectric sounder are not in direct contact with each other.

9. The module according to claim 1,
wherein the biological information obtainer includes at least one of a heart rate sensor, a blood pressure sensor, and a blood oxygenation sensor.

10. The module according to claim 2,
wherein the biological information obtainer includes at least one of a heart rate sensor, a blood pressure sensor, and a blood oxygenation sensor.

11. The module according to claim 3,
wherein the biological information obtainer includes at least one of a heart rate sensor, a blood pressure sensor, and a blood oxygenation sensor.

12. The module according to claim 4,
wherein the biological information obtainer includes at least one of a heart rate sensor, a blood pressure sensor, and a blood oxygenation sensor.

13. A wearable device comprising the module according to claim 1.

14. A wearable device comprising the module according to claim 2.

15. A wearable device comprising the module according to claim 3.

16. A wearable device comprising the module according to claim 4.

17. The wearable device according to claim 13, further comprising:
a device case that holds the circuit board and the piezoelectric sounder.

18. The wearable device according to claim 17, further comprising:
a case back on a side of the device case, the side facing a living body from which biological information is obtained.

19. The wearable device according to claim 18,
wherein the circuit board is fixed to a predetermined position on the case back.

20. The wearable device according to claim 19,
wherein at least a part of a piezoelectric body constituting the piezoelectric sounder overlaps a fixing surface at which a metal plate to which the piezoelectric body is adhered is fixed to the case back.
